(19) United States
(12) Patent Application Publication
(10) Pub. No.: US 2010/0035805 A1
(43) Pub. Date: Feb. 11, 2010

(54) NON-AQUEOUS LIQUID FORMULATION FOR NASAL OR BUCCAL ADMINISTRATION

(75) Inventor: Peter Roderick Hafner, Wiltshire (GB)

Correspondence Address: PROSKAUER ROSE LLP, One International Place, Boston, MA, 02110, US

(73) Assignee: OptiNose AS, Oslo (NO)

(21) Appl. No.: 12/298,292

(22) PCT Filed: Apr. 25, 2007
(86) PCT No.: PCT/GB2007/001515

§ 371 Date: Jul. 2, 2009

(30) Foreign Application Priority Data
Apr. 25, 2006 (GB) 06080980.0

(51) Int. Cl. A61K 38/31; A61K 38/08; A61K 38/11; A61K 38/22; A61P 5/10; A61P 5/04

(52) U.S. Cl. 514/12; 514/17; 514/15

(57) ABSTRACT

A formulation for administration to a nasal or buccal cavity of a subject which comprises a non-aqueous liquid environment, preferably an emollient oil base, and at least one active molecule, preferably a dopamine agonist and especially apomorphine, in solution or suspension therein, wherein in one embodiment the formulation has such a viscosity as to be delivered as a liquid jet from a spray pump which is capable of delivering an aerosol spray of an aqueous formulation.

The present invention relates to a liquid formulation, and in particular a formulation for administration to the nasal or buccal cavities of a subject.

In a preferred embodiment the present invention relates to a formulation which contains a dopamine agonist, such as apomorphine, for administration to the nasal cavity of a subject, and in particular for the treatment of breakthrough dyskinesia and sexual dysfunction.

A particular problem associated with formulations which contain apomorphine is the sensitivity of apomorphine to oxidation.

Existing formulations attempt to address this problem through the use of anti-oxidants, such as sodium metabisulphite, sodium ascorbate and ascorbic acid, and regulating the pH of the formulation to an acidic pH, and typically at a pH of 3.

It is one aim of the present invention to provide a formulation which allows for a relatively-high concentration of the active molecule in solution or suspension and in turn provides a relatively high bio-availability.

It is another aim of the present invention to provide a formulation which provides sufficient resistance to auto-oxidation and avoids the need for the use a low pH and harsh anti-oxidants. Some clinical studies have reported nasal irritation, which could be caused by the use of a low pH and harsh anti-oxidants.

It is a further aim of the present invention to provide a formulation which allows for the delivery of a liquid jet when using a standard spray pump, which can be advantageous is targeting specific regions in the nasal or buccal cavities, in particular the posterior region of the nasal cavity.

It is a still further aim of the present invention to provide a non-aqueous formulation which allows for the delivery of a liquid spray when using a standard spray pump.

In one aspect the present invention provides a formulation for administration to the nasal or buccal cavities of a subject, preferably for administration using a pump mechanism, typically a conventional spray pump, which comprises a non-aqueous liquid environment or carrier and at least one active molecule.

Preferably, the non-aqueous liquid environment comprises an emollient oil base.

In one embodiment the emollient oil base includes at least one of a vegetable, mineral or animal oil.

In one embodiment the emollient oil base comprises a vegetable oil,

Preferably, the vegetable oil comprises at least one of almond oil, anise oil, apricot kernel oil, arachis oil, argan oil, avocado oil, borage oil, cajuput oil, canola oil, caraway oil, cassia oil, castor oil, cinnamon oil, citronella oil, clove oil, coconut oil, coriander oil, corn oil, cottonseed oil, eucalyptus oil, evening primrose oil, fennel oil, geranium oil, grapeseed oil, hazelnut oil, hemp oil, jojoba oil, juniper oil, lavender oil, lemon oil, macadamia oil, mace oil, melaleuca oil, neem oil, neroli oil, niaouli oil, nutmeg oil, olive oil, orange oil, palm oil, palm kernel oil, pine oil, poppyseed oil, pulegium oil, pumpkin seed oil, rapeseed oil, rice bran oil, rosehip oil, rosemary oil, rue oil, safflower oil, sesame oil, spearmint oil, soybean oil, sunflower oil, thyme oil, walnut oil and wheatgerm oil.

In another embodiment the emollient oil base includes a fatty acid.

In a further embodiment the emollient oil base includes at least one of a monoglyceride, diglyceride or triglyceride.

In one embodiment the at least one active molecule is a drug substance which degrades when in aqueous solution, such as by way of oxidation or hydrolysis.

In one embodiment the at least one active molecule comprises a small organic molecule, and preferably an organic molecule having a molecular weight of less than 1000.

In one embodiment the at least one active molecule comprises a dopamine agonist.

Preferably, the dopamine agonist comprises apomorphine or its pharmaceutically-acceptable derivatives or analogues.

In one embodiment the at least one active molecule comprises a drug which is stored as a lyophilisate or normally required to be refrigerated.

In one embodiment the at least one active molecule is a protein or a peptide.

In one embodiment the at least one active molecule comprises an antidiurectic hormone, such as argipressin, lypressin, desmopressin, felypressin, ornipressin, terlipressin and vasopressin or their pharmaceutically-acceptable derivatives or analogues.

In one embodiment the at least one active molecule comprises an oxytocic hormone, such as carbetocin, demoxytocin and oxytocin or their pharmaceutically-acceptable derivatives or analogues.

In one embodiment the at least one active molecule comprises an oxytocin antagonist, such as atosiban or its pharmaceutically-acceptable derivatives or analogues.

In one embodiment the at least one active molecule comprises a corticotrophic hormone, such as corticotrophin and tetracosactide or their pharmaceutically-acceptable derivatives or analogues.

In one embodiment the at least one active molecule comprises a corticotrophic releasing hormone, such as corticorelin or its pharmaceutically-acceptable derivatives or analogues.

In one embodiment the at least one active molecule comprises an omatotrophic hormone, such as mecasermin, somtrem and somatropin or their pharmaceutically-acceptable derivatives or analogues.

In one embodiment the at least one active molecule comprises a somatotrophic hormone receptor antagonist, such as pegvisomant or its pharmaceutically-acceptable derivatives or analogues.

In one embodiment the at least one active molecule comprises an omatotrophic releasing hormone, such as sermorelin and somatorelin or their pharmaceutically-acceptable derivatives or analogues.

In one embodiment the at least one active molecule comprises a somatotrophic release inhibitor, such as lanreotide, octreotide, somatostatin and vapreotide or their pharmaceutically-acceptable derivatives or analogues.

In one embodiment the at least one active molecule comprises a gonadotrophic hormone, such as choriogonadotrophin alfa, chorionic gonadotrophin, a follicle stimulating hormone, follitropin alfa, follitropin beta, a luteinising hormone, lutropin alfa, menotrophin and urofollitropin or their pharmaceutically-acceptable derivatives or analogues.

In one embodiment the at least one active molecule comprises a gonadotrophic releasing hormone, such as buserelin, deslorelin, gonadorelin, goserelin, histrelin, leuprorelin, naferlin and triptorelin or their pharmaceutically-acceptable derivatives or analogues.

In one embodiment the at least one active molecule comprises an onadotrophic releasing hormone antagonist, such as abarelix, cetorelix and ganirelix or their pharmaceutically-acceptable derivatives or analogues.

In one embodiment the at least one active molecule comprises a thyrotrophic hormone, such as thyrotrophin and thyrotrophin alfa or their pharmaceutically-acceptable derivatives or analogues.

In one embodiment the at least one active molecule comprises a thyrotrophic releasing hormone, such as posatirelin, protirelin and taltirelin or their pharmaceutically-acceptable derivatives or analogues.

In one embodiment the at least one active molecule comprises a lactotrophic hormone, such as prolactin or its pharmaceutically-acceptable derivatives or analogues.

In one embodiment the at least one active molecule comprises a metabolic peptide, such as insulin, an insulin-like growth factor, a glucagon, a growth hormone and PYY3-36 or their pharmaceutically-acceptable derivatives or analogues.

In one embodiment the at least one active molecule comprises calcitonin or pharmaceutically-acceptable derivatives or analogues thereof, such as elcatonin and salcatonin.

In one embodiment the at least one active molecule comprises a melanocyte stimulating hormone.

In one embodiment the at least one active molecule comprises a nerve growth factor.

In one embodiment the at least one active molecule comprises an epidermal growth factor.

In one embodiment the at least one active molecule comprises an epoetin or its pharmaceutically-acceptable derivatives or analogues.

In one embodiment the at least one active molecule comprises an interleukin.

In one embodiment the at least one active molecule comprises a protein involved in one or both of blood coagulation and fibrinolysis.

In one embodiment the at least one active molecule comprises an antibiotic, such as lactams, penicillins and cephalosporins.

In one embodiment the at least one active molecule comprises water-labile esters, such as aspirin or its pharmaceutically-acceptable analogue or derivative.

In one embodiment the at least one active molecule comprises benzocain or its pharmaceutically-acceptable analogue or derivative.

In one embodiment the at least one active molecule comprises N-acetyl p-aminophenol or its pharmaceutically-acceptable analogue or derivative.

In one embodiment the at least one active molecule comprises a prostaglandin analogue or derivative.

In one embodiment the at least one active molecule comprises indole-3-carbinol (I3C) or its pharmaceutically-acceptable analogue or derivative.

In one embodiment the at least one active molecule comprises water-labile amides.

Preferably, the formulation is substantially free of anti-microbial preservative.

In one embodiment the at least one active molecule is in solution in the non-aqueous liquid environment.

In another embodiment the at least one active molecule is in suspension in the non-aqueous liquid environment.

In one embodiment the viscosity of the formulation is such as to be delivered as a liquid jet from a spray pump which is capable of delivering an aerosol spray of an aqueous formulation.

In another embodiment the viscosity of the formulation is such as to be delivered as a liquid spray from a spray pump which is capable of delivering an aerosol spray of an aqueous formulation.

In one embodiment the formulation comprises a nasal formulation for administration to the nasal cavity.

In another embodiment the formulation comprises a buccal formulation for administration to the buccal cavity, such as sub-lingual administration.

In another aspect the present invention relates to a method of treating breakthrough dyskinesia or sexual dysfunction, which comprises the step of intranasally administering the above-described formulation to a nasal airway of a subject.

In one embodiment the formulation is delivered as a liquid jet.

In another embodiment the formulation is delivered as a liquid spray.

A preferred embodiment of the present invention will now be described hereinbelow by way of example only with reference to the accompanying drawings, in which:

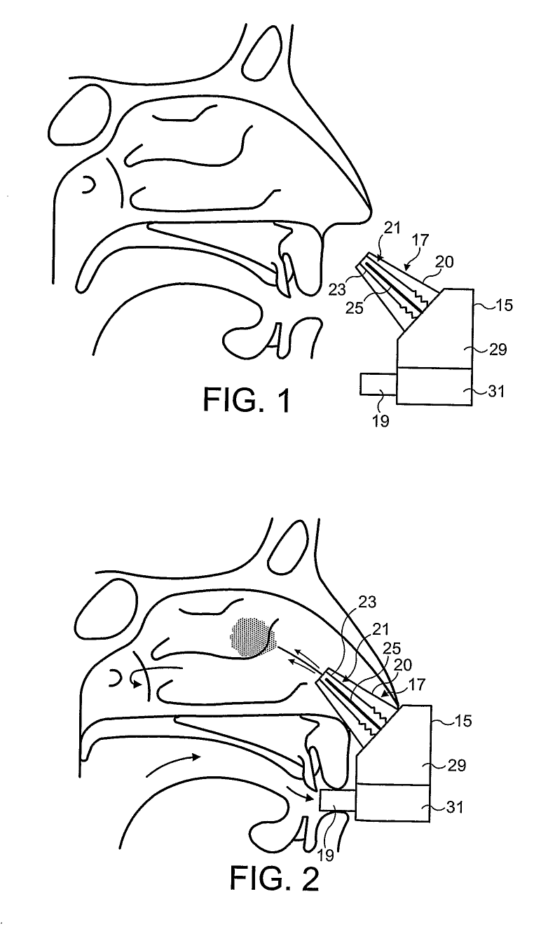

FIG. 1 schematically illustrates a nasal delivery device for delivering a formulation to a nasal airway of a subject in accordance with one embodiment of the present invention; and

FIG. 2 illustrates the delivery device of FIG. 1 where operative to deliver a dose of the embodied formulation into the nasal airway of the subject.

The delivery device provides for the delivery of a nasal formulation which comprises an emollient oil base and apomorphine. In this embodiment the apomorphine is in solution in the emollient oil base.

As will be described further hereinbelow, in this embodiment the formulation has such a viscosity as to be delivered as a liquid jet from a spray pump which is capable of delivering an aerosol spray of an aqueous formulation.

The delivery device comprises a housing 15, a nosepiece unit 17 for fitting in a nasal cavity of a subject, and a mouthpiece 19 through which the subject exhales to actuate the delivery device.

The nosepiece unit 17 comprises a nosepiece 20, in this embodiment a frusto-conical element, for guiding the nosepiece unit 17 into a nasal cavity of the subject, and an outlet unit 21 for delivering the nasal formulation into the nasal airway of the subject.

In this embodiment the outlet unit 21 comprises a delivery channel 23 which is in fluid communication with the mouthpiece 19 such that an air flow is delivered into and through the nasal airway of the subject on exhalation by the subject through the mouthpiece 19, and a nozzle 25 for delivering the nasal formulation to the nasal airway of the subject.

The nozzle 25 is of a kind which is capable of delivering an aerosol spray, but is such as to deliver the nasal formulation of the present invention, as a liquid jet, that is, as a column of liquid.

The delivery device further comprises a pump unit 29 for delivering metered doses of the formulation, which is fluidly connected to the nozzle 25 to deliver the nasal formulation from the nosepiece 17, in this embodiment as a liquid jet.

In this embodiment the pump unit 29 is a multi-dose unit for delivering a plurality of metered doses of the nasal formulation. In another embodiment the pump unit 29 could be a single-dose unit for delivering a single metered dose of the nasal formulation.

The pump unit 29 is pre-primeable, in this embodiment by loading a resilient element, and includes a breath-actuated release mechanism 31 which, when triggered, releases the resilient element and actuates the pump unit 29 to deliver a metered dose of the nasal formulation through the nozzle 25.

In this embodiment the trigger mechanism 31 is configured to cause actuation of the pump unit 29 on generation of a predetermined flow rate through the delivery channel 23.

Operation of the delivery device will now be described hereinbelow with reference to FIG. 2 of the accompanying drawings.

The nosepiece 17 is first inserted into one of the nasal cavities of a subject until the nosepiece 20 abuts the nares of the nostril, at which point the distal end of the outlet unit 21 extends about 2 cm into the nasal cavity of the subject, and the mouthpiece 19 is gripped in the lips of the subject.

The subject then begins to exhale through the mouthpiece 19, which exhalation acts to close the oropharyngeal velum of the subject and drive an air flow through the delivery channel 23 of the outlet unit 21, with the air flow passing into the one nasal cavity, around the posterior margin of the nasal septum and out of the other nasal cavity, thereby achieving a bi-directional air flow through the nasal airway of the subject.

In this embodiment, when the flow rate developed through the delivery channel 23 reaches a predetermined value, the release mechanism 31 is triggered to actuate the pump unit 29 to deliver a metered dose of the nasal formulation to the nozzle 25 and into the nasal cavity of the subject as a liquid jet.

In this embodiment, where the delivery device is a multi-dose device, the device is ready for further use following priming of the pump unit 29.

In an alternative embodiment the formulation can be formulated such as to be delivered as a liquid spray. In one embodiment the non-aqueous, oil base can have a viscosity which does not allow for the delivery of a spray, and in this embodiment the formulation can include a thinning agent which acts to reduce the viscosity of the formulation to allow for the delivery of a spray.

### EXAMPLE

In order to exemplify the present invention, the present invention will now be described hereinbelow with reference to the following non-limiting Example.

Two samples were prepared by adding 500 mg of apomorphine HCI to both 40 ml of sesame seed oil (Ph. Eur.) and 40 ml of water. These samples were shaken to ensure a saturated solution.

The auto-oxidation of apomorphine can be detected by the development of a green colour, which represents the oxidation product.

In the water-based sample, a green colour was observed within an hour, a dark green colour was observed within four hours and the sample had an intense green colour after three days.

In the oil-based sample, no green colour was observed within three days, and the oil maintained the characteristic yellow colour of the sesame seed oil for a period of at least six months.

The oil-based sample was also delivered from a conventional spray pump, and the sample was delivered as a liquid jet as opposed to an aerosol spray.

Finally, it will be understood that the present invention has been described in its preferred embodiment and can be modified in many different ways without departing from the scope of the invention as defined by the appended claims.

In the above-described embodiment the delivery device is configured to deliver an air flow through one nostril of a subject at such a pressure as to flow around the posterior margin of the nasal septum and out of the other nostril of the subject, thereby achieving bidirectional delivery through the nasal cavities as disclosed in WO-A-00/51672, the content of which is herein incorporated by reference, but in an alternative embodiment the delivery device could be configured to deliver an air flow which is not sufficient to achieve bi-directional delivery through the nasal cavities or utilizes no entraining gas flow. This embodiment is still advantageous as compared to known delivery devices, in providing for velum closure and being capable of achieving targeted delivery, particularly when certain regions of the nasal cavity are obstructed by cuff members.

In another embodiment the above-described delivery device can be modified such as to omit the mouthpiece 19 and provide for manual actuation of the trigger mechanism 31, in which case the delivery device acts in the manner of a conventional spray device, but where delivering the formulation as a liquid jet.

In still another embodiment, with the delivery device so modified, the delivery device can provide for administration to the buccal cavity, for example, for sub-lingual administration.

1. A formulation which comprises a non-aqueous liquid environment and at least one active molecule for administration to a nasal or buccal cavity of a subject.

2. The formulation of claim 1, wherein the non-aqueous liquid environment comprises an emollient oil base.

3. The formulation of claim 2, wherein the emollient oil base includes at least one of a vegetable, mineral or animal oil, preferably the emollient oil base comprises a vegetable oil, and preferably the vegetable oil comprises at least one of almond oil, anise oil, apricot kernel oil, arachis oil, argan oil, avocado oil, borage oil, cajuput oil, canola oil, caraway oil, cassia oil, castor oil, cinnamon oil, citronella oil, clove oil, coconut oil, coriander oil, corn oil, cottonseed oil, eucalyptus oil, evening primrose oil, fennel oil, geranium oil, grapeseed oil, hazelnut oil, hemp oil, jojoba oil, juniper oil, lavender oil, lemon oil, macadamia oil, mace oil, melaleuca oil, neem oil, neroli oil, niaouli oil, nutmeg oil, olive oil, orange oil, palm oil, palm kernel oil, pine oil, poppyseed oil, pulegium oil, pumpkin seed oil, rapeseed oil, rice bran oil, rosehin oil rosemary oil, rue oil, safflower oil, sesame oil, spearmint oil, sovbean oil, sunflower oil, thyme oil, walnut oil and wheatgerm oil.

4. (canceled)

5. (canceled)

6. The formulation of claim 2, wherein the emollient oil base includes a fatty acid.

7. The formulation of claim 2, wherein the emollient oil base includes at least one of a monoglyceride, diglyceride or triglyceride.

8. The formulation of claim 1, wherein the at least one active molecule is a drug substance which degrades when in aqueous solution preferably the degradation is one of oxidation or hydrolysis.

9. (canceled)

10. The formulation of claim 8, wherein the at least one active molecule comprises an organic molecule with a molecular weight of less than about 1000.

11. The formulation of claim 8, wherein the at least one active molecule comprises a dopamine agonist, preferably the dopamine agonist comprises apomorphine or a pharmaceutically-acceptable derivative or analogue thereof.

12. (canceled)

13. The formulation of claim 8, wherein the at least one active molecule is a protein or a peptide.

14. The formulation of claim 13, wherein the at least one active molecule comprises an antidiurectic hormone, such as argipressin, lypressin, desmopressin, felypressin, ornipressin, terlipressin and vasopressin or a pharmaceutically-acceptable derivative or analogue thereof, an oxytocic hormone, such as carbetocin, demoxytocin and oxytocin or a pharmaceutically-acceptable derivative or analogue thereof, an oxytocin antagonist, such as atosiban or a pharmaceutically-acceptable derivative or analogue thereof, a corticotrophic hormone, such as corticotrophin and tetracosactide or a pharmaceutically-acceptable derivative or analogue thereof, a corticotrophic releasing hormone, such as corticorelin or a pharmaceutically-acceptable derivative or analogue thereof, an omatotrophic hormone, such as mecasermin, somtrem and somatropin or a pharmaceutically-acceptable derivative or analogue thereof, a somatotrophic hormone receptor antagonist, such as pegvisomant or a pharmaceutically-acceptable derivative or analogue thereof, an omatotrophic releasing hormone, such as sermorelin and somatorelin or a pharmaceutically-acceptable derivative or analogue thereof, a somatotrophic release inhibitor, such as lanreotide, octreotide, somatostatin and vapreotide or a pharmaceutically-acceptable derivative or analogue thereof a gonadotrophic hormone, such as choriogonadotrophin alfa, chorionic gonadotrophin, a follicle stimulating hormone, follitropin alfa, follitropin beta, a luteinising hormone, lutropin alfa, menotrophin and urofollitropin or a pharmaceutically-acceptable derivative or analogue thereof, a gonadotrophic releasing hormone, such as buserelin, deslorelin, gonadorelin, goserelin, histrelin, leuprorelin, naferlin and triptorelin or a pharmaceutically-acceptable derivative or analogue thereof, an onadotrophic releasing hormone antagonist, such as abarelix, cetorelix and ganirelix or a pharmaceutically-acceptable derivative or analogue thereof, a thyrotrophic hormone, such as thyrotrophin and thyrotrophin alfa or a pharmaceutically-acceptable derivative or analogue thereof, a thyrotrophic releasing hormone, such as posatirelin, protirelin and taltirelin or a pharmaceutically-acceptable derivative or analogue thereof, a lactotrophic hormone, such as prolactin or a pharmaceutically-acceptable derivative or analogue thereof, a metabolic peptide, such as insulin, an insulin-like growth factor, a glucagon, a growth hormone and PYY3-36 or a pharmaceutically-acceptable derivative or analogue thereof, a calcitonin or a pharmaceutically-acceptable derivative or analogue thereof, such as elcatonin and salcatonin, a melanocyte stimulating hormone, a nerve growth factor, an epidermal growth factor, an epoetin or a pharmaceutically-acceptable derivative or analogue thereof, an interleukin, a protein involved in one or both of blood coagulation and fibrinolysis, or an antibiotic, such as lactams, penicillins and cephalosporins.

15-37. (canceled)

38. The formulation of claim 8, wherein the at least one active molecule comprises water-labile esters, such as aspirin or a pharmaceutically-acceptable derivative or analogue thereof.

39. The formulation of claim 8, wherein the at least one active molecule comprises benzocain or a pharmaceutically-acceptable derivative or analogue thereof.

40. The formulation of claim 8, wherein the at least one active molecule comprises N-acetyl p-aminophenol or a pharmaceutically-acceptable derivative or analogue thereof.

41. The formulation of claim 8, wherein the at least one active molecule comprises a prostaglandin analogue or derivative.

42. The formulation of claim 8, wherein the at least one active molecule comprises indole-3-carbinol (I3C) or a pharmaceutically-acceptable derivative or analogue thereof.

43. The formulation of claim 8, wherein the at least one active molecule comprises water-labile amides.

44. The formulation of claim 1, wherein the formulation is substantially free of anti-microbial preservative.

45. The formulation of any of claim 1, wherein the at least one active molecule is in solution or suspension in the non-aqueous liquid environment.

46. (canceled)

47. The formulation of claim 1, wherein the viscosity of the formulation is such as to be delivered as a liquid jet from a spray pump which is capable of delivering an aerosol spray of an aqueous formulation or as a liquid spray from a spray pump which is capable of delivering an aerosol spray of an aqueous formulation.

48. (canceled)

49. The formulation of claim 1, wherein the formulation comprises a nasal formulation for administration to the nasal cavity.

50. The formulation of claim 1, wherein the formulation comprises a buccal formulation for administration to the buccal cavity preferably for sub-lingual administration.

51. (canceled)

52. A method of treating breakthrough dyskinesia or sexual dysfunction, which comprises the step of intranasally administering the formulation of claim 49 to a nasal airway of a subject.

53. The method of claim 52, wherein the formulation is delivered as a liquid jet or a liquid spray.

54-58. (canceled)
